(19) United States
(12) Patent Application Publication
(10) Pub. No.: US 2026/0315022 A1
(43) Pub. Date: Oct. 15, 2026

(54) X-RAY SYSTEM FOR THE PRODUCTION OF DENTAL PANORAMIC IMAGES WITH SEVERAL OFFSET INDIVIDUAL X-RAY EMITTERS

(71) Applicant: DENTSPLY SIRONA Inc., York, PA (US)

(72) Inventors: Stefan Eichner, Heidelberg (DE); Susanne Maur, Bensheim (DE)

(21) Appl. No.: 19/107,479

(22) PCT Filed: Aug. 29, 2023
(86) PCT No.: PCT/EP2023/073604

§ 371 Date: Feb. 27, 2025

(30) Foreign Application Priority Data
Aug. 29, 2022 (EP) 22192554.8

(51) Int. Cl. A61B 6/51; A61B 6/00; A61B 6/06; A61B 6/40

(52) U.S. Cl. CPC A61B 6/51; A61B 6/06; A61B 6/4007; A61B 6/4452; A61B 6/5205; A61B 6/54

(57) ABSTRACT

The present invention relates to an extraoral x-ray system (1) suitable for dental panoramic imaging of a patient, comprising: an x-ray emitter array (3) having at least two individual x-ray emitters (3a; 3b; 3c) each for emitting x-rays, and offset at least along a predetermined direction; and an x-ray detector (4) for at least partially detecting the x-rays emitted by the individual x-ray emitters (3a; 3b; 3c) during one revolution, wherein the x-ray emitter array (3) and the x-ray detector (4) being arranged for movement relative to one another about an axis running parallel to the said predetermined direction, wherein the areas of the x-ray detector (4) respectively irradiated by the individual x-ray emitters (3a; 3b; 3c) at least partially overlap; an aperture means for collimating the x-ray radiation emitted by the individual x-ray emitters (3a; 3b; 3c) to the respective areas to be irradiated; a control device for moving the x-ray emitter array (3) and the x-ray detector (4) about the said parallel running axis, and for controlling the individual x-ray emitters (3a; 3b; 3c) and for reading out the image series of the respective irradiated areas of the X-ray detector (4) during the said revolution; a computing unit for generating one panoramic image (16) of a layer to be sharply imaged by using said at least two image series, the computing unit being configured to apply a weighting of image pixels when generating the said one panoramic image (16), wherein the said image pixels belong to at least the two image series.

### TECHNICAL FIELD OF THE INVENTION

The present invention relates to an x-ray system for producing orthopantomography images in the dental field.

### BACKGROUND OF THE INVENTION

During an orthopantomography or panoramic imaging (PAN), the x-ray emitter and the x-ray detector of an extraoral x-ray system generally move around a patient's head in defined relative positions to each other. This can lead to unfavorable superimposition of the imaged structures, which can complicate the diagnosis and may require repeat exposures. In PAN, movement around the patient's head during imaging results in horizontal blurring of structures outside the sharp layer. The sharp layer defines a layer that preferably runs along the jaw arch, and which has the dental structures to be imaged, and is preferably displayed with good image quality in the panoramic image. For the production of the panoramic image, for example, a “Shift & Add” procedure can be used which sums the projection images of the exposure. Nevertheless, structures lying in the x-ray path create shadowing in the sharp layer and reduce the contrast in the panoramic image. For example, fillings in the current pivot point of the x-ray emitter and x-ray detector unit lead to strong artifacts in the opposing jaw (see example in FIG. 2). These are commonly referred to as opposing jaw artifacts in the prior art. For example, the tooth crown (13) causes an opposing jaw artifact (12) in the representation of the opposite side of the jaw in the panoramic image. Similarly, the jawbone (15), for example, causes an opposing jaw artifact (12′) in the representation of the opposing jaw side in the panoramic image.

In the prior art, this problem is mitigated as follows: A different imaging program of the extraoral x-ray system is selected to apply a different x-ray device trajectory for the panoramic imaging. Alternatively, the position and/or orientation of the patient's head in the x-ray device is adjusted to reduce the superimposition of interfering structures and thus opposing jaw artifacts.

In the prior art, the problem cannot be adequately solved, and thus not all artifacts can be reduced. Repeat exposures may be required, resulting in increased radiation exposure to the patient.

In addition, the prior art approach to patient re-positioning and/or imaging program re-selection results in complicated and error-prone x-ray device operation and potentially reduced image quality, so that this approach requires a great deal of experience on the part of the x-ray device operator.

EP3649957A1 and EP3711672B respectively disclose devices and methods for processing a panoramic image, wherein opposing jaw artifacts are reduced by software.

### DISCLOSURE OF THE INVENTION

The inventors are currently not aware of any prior art method that enables the reduction of opposing jaw artifacts by vertically blurring the structures outside the sharp layer. Whereby the vertical blurring causes a vertical unsharpness of the structures to be imaged.

An objective of the present invention is to provide a PAN-enabled X-ray system that allows reduction of opposing jaw artifacts by vertically blurring structures outside the sharp layer.

This objective has been achieved by the PAN-enabled X-ray system according to claim 1. The subject-matter of the dependent claims relate to preferred embodiments or further developments.

The invention provides an extraoral x-ray system suitable for a panoramic dental imaging procedure of a patient. The extraoral x-ray system comprises: an x-ray emitter array having at least two individual x-ray emitters each for emitting x-rays, which are offset at least along a predetermined direction (e.g., a patient longitudinal direction or a height/vertical direction); and an x-ray detector for at least partially detecting the x-ray radiations emitted by the individual x-ray emitters during one revolution, wherein the x-ray emitter array and the x-ray detector are disposed movably relative to one another about an axis running parallel to the said predetermined direction, wherein the areas of the x-ray detector which are respectively irradiated by the individual x-ray emitters at least partially overlap; an aperture means for collimating the x-ray radiations emitted by the individual x-ray emitters onto the respective areas to be irradiated; a control device for moving the x-ray emitter array and the x-ray detector about the parallel running axis, and for driving the individual x-ray emitters and for reading out the image series of the respectively irradiated areas of the x-ray detector during the said revolution; a computing unit for generating one panoramic image of a layer to be sharply imaged by using the at least two said image series, wherein the computing unit is configured in such a way that a weighting of image pixels is applied when generating the said one panoramic image, wherein the said image pixels belong at least to the two image series.

An advantageous effect of the present invention is that the offset or height-shifted single emitters result in different transmission angles, so that the corresponding detector signals enable the separation of the overlapping structures in the reconstructed image. In particular, due to the multiple available viewing angles of the height-shifted single emitters, an improved representation of the structures within the sharp layer can be achieved, mainly because the structures outside the sharp layer can be mapped to different positions in the reconstructed panoramic image and can be blurred more. Weighting the image pixels during the reconstruction of the panoramic image can additionally increase the contrast of the structures inside the sharp layer and additionally decrease the contrast of the structures outside the sharp layer. This allows for improved visualization of smaller areas such as caries, lesions, roots, or nerve canals, and diagnostic discoverability. Also, by weighting the image pixels, those structures can be specifically suppressed which cause strong opposing jaw artifacts. The resulting reduction of opposing jaw artifacts in the panoramic image enables simplified x-ray device operation, as possible opposing jaw artifacts no longer need to be taken into account when positioning the patient and selecting the x-ray device trajectory and imaging program. This also allows the use of more suitable transmission angles and thus also contributes to improved image quality. For example, in the area of the anterior teeth, this enables significantly improved imaging, especially in the case of unfavorable tooth positions, such as a large difference in the tooth axes between the upper and lower jaws. The present invention also makes it possible to determine whether imaged structures lie inside or outside the sharp layer. For this purpose, image pixels of the multiple individual x-ray emitters can be compared. If these show different absorption in the reconstructed panoramic image, they are images of structures outside the sharp layer.

### BRIEF DESCRIPTION OF THE DRAWINGS

In the following description, the present invention will be explained in more detail by means of exemplary embodiments and with reference to the drawings, wherein

FIG. 1—shows an extraoral x-ray system according to one embodiment of the present invention;

FIG. 2—shows an example image of strong opposing jaw artifacts according to the state of the art;

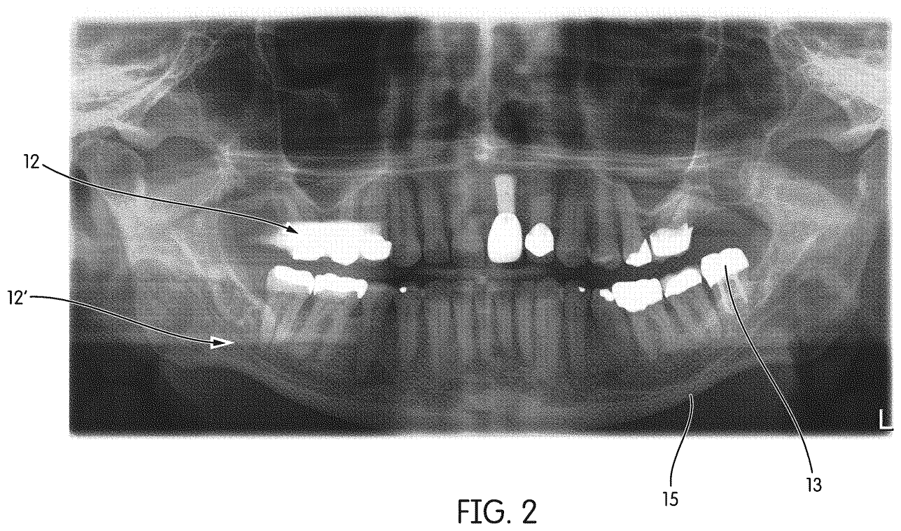

FIGS. 3a, 3b—show two panoramic images, each reconstructed from the two image series of the two single x-ray emitters with different positions and characteristics of the opposing jaw artifacts;

FIG. 4—shows a schematic detailed representation of the setup of the x-ray emitter array with locally distributed individual x-ray emitters in the extraoral X-ray system from FIG. 1.

The reference numbers shown in the drawings designate the elements listed below, which are referred to in the following description of the exemplary embodiments.
1. Extraoral X-ray system
2. X-ray device
3. X-Ray Array
3a,3b,3c. Single x-ray emitter
4. X-ray detector
5. Operating unit
6. Head fixation
7. Bite block
8. Computer
9. Display
10. Patient head
11. Spotlight cone
12. 12′, 12″, 12″ Counter jaw artifact
13. Tooth crown
14. Earring
15. 15′ Jaw bone
16. Dental panoramic image

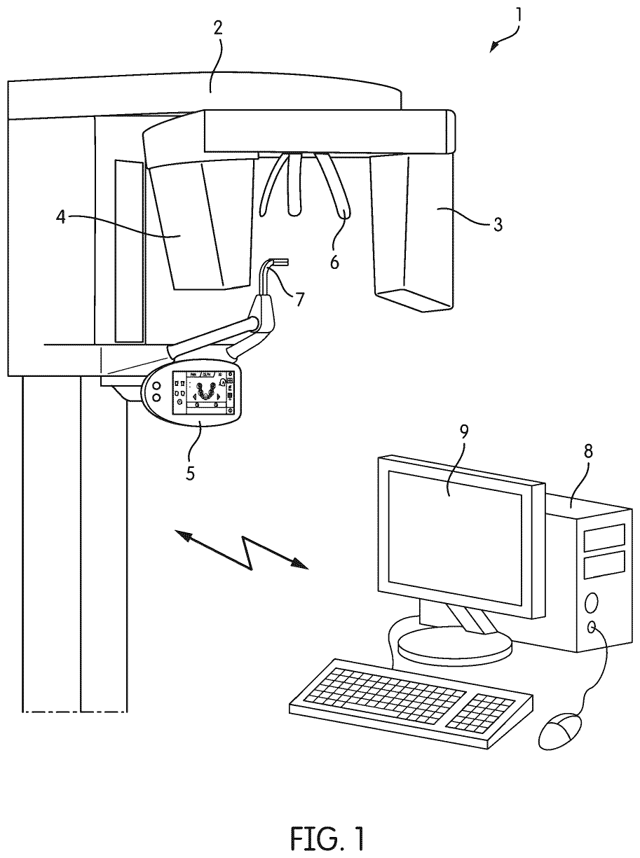

FIG. 1 shows an embodiment of the computer-implemented extraoral x-ray system (1). The method according to the invention is a computer-implemented method, and can be executed on a computer-implemented extraoral x-ray system (1). The method according to the invention is implemented by a computer program comprising computer-readable code. The computer program may be provided on a data storage device. As shown in FIG. 1, the computerized extraoral X-ray system (1) comprises an x-ray device (2) for performing the patient imaging. Prior to the exposure, the patient head is preferably positioned in the x-ray device (2) with the bite block (7) and the head fixation (6). As shown in FIG. 1, the computerized extraoral X-ray system (1) has an operating unit (5), preferably a separate computer (8) or computing unit that can be connected to the x-ray device (2), and preferably a separate display (9), inter alia to visualize data sets. The computer (8) can be connected to the x-ray device (2) via a local network (not shown) or alternatively via the Internet. The computer (8) may be part of a cloud. Alternatively, the computer (8) may be integrated into the x-ray device (2). The calculations can take place in the computer (8) or in the cloud. For this purpose, the raw data can be transmitted in compressed form. The computer (8) executes the computer program and provides the data sets, including for the visualization on the display (9). The display (9) can be spatially separated from the x-ray device (2). Preferably, the computer (8) can also control the x-ray device (2). Alternatively, separate computers (8) may be used for the control and image processing. According to the present invention, the data sets generated by the above embodiment may be presented to a physician for visualization, in particular for diagnostic purposes, preferably by means of the display (9) or a printout.

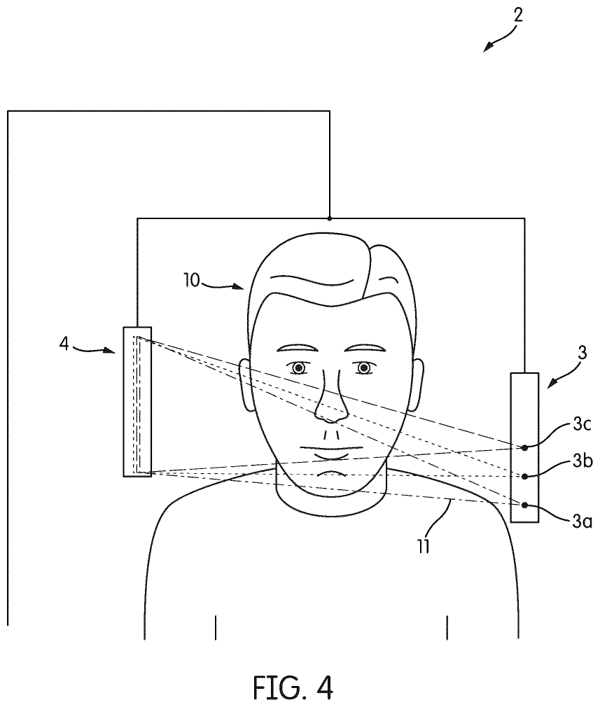

The extraoral x-ray system (1) is suitable for performing the dental panoramic imaging method. As shown in FIG. 1, the x-ray system (1) comprises an x-ray emitter array (3) having at least two individual x-ray emitters (3a;3b;3c) each for emitting x-ray radiation (see FIG. 4), which are offset at least along a predetermined direction (e.g., in the height direction or longitudinal direction of the patient). The x-ray system (1) further comprises an x-ray detector (4) for at least partially detecting the x-ray radiations emitted by the individual x-ray emitters (3a;3b;3c) during one revolution, wherein the x-ray emitter array (3) and the x-ray detector (4) are arranged movably relative to one another about an axis running parallel to said predetermined direction, wherein the areas of the x-ray detector (4) respectively irradiated by the individual x-ray emitters (3a;3b;3c) at least partially overlap. The x-ray system (1) further comprises an aperture means (not shown) for collimating the x-ray radiation emitted by the individual x-ray emitters (3a;3b;3c) onto the said respective areas to be irradiated. The x-ray system (1) further comprises a control device (not shown) for moving the x-ray emitter array (3) and the X-ray detector (4) around the parallel axis, and for controlling the individual x-ray emitters (3a;3b;3c) and for reading out the image series of the respective irradiated surfaces of the X-ray detector (4) during the rotation. The x-ray system (1) further comprises a computing unit (8) for generating one panoramic image (16) of a layer to be sharply imaged by using the at least two said image series, wherein the computing unit is configured (programmed) to apply a weighting of image pixels when generating the said one panoramic image (16), wherein the said image pixels belong to at least the two image series.

In an advantageous embodiment, the total height of the irradiated areas along the predetermined direction is greater than the total width of the irradiated areas along a direction orthogonal thereto by a factor of at least F=3 times. In a further advantageous embodiment, the factor F is in the range 15 to 25, preferably about 22. The total height of the irradiated areas and the total width of the irradiated areas correspond to the effective height and width on the x-ray detector. Whereby the areas irradiated by each of the individual emitters may overlap in height and/or width. Whereby the areas respectively irradiated by the individual emitters can preferably partially overlap in height and can preferably almost completely overlap in width. The factor F leads to a smaller size of the irradiated total area while the height remains the same. This has the advantage of enabling a high readout rate of the x-ray detector with low motion blurring, as well as causing a low proportion of measured scattered radiation on the x-ray detector, which improves the image quality. The lower irradiated area also reduces the applied dose to the patient.

During one revolution, the x-ray emitter array and x-ray detector move around the patient, with the x-ray emitter array emitting x-rays and the x-ray detector detecting the emitted x-rays. For each individual x-ray emitter of the x-ray emitter array, a series of images can be generated during the revolution from the detected x-rays.

In a further embodiment, the operating state of the x-ray emitter array (or the x-ray device) is varied during the revolution such that the position of one of the focal points varies along the predetermined direction. Where the focal points describe the locations within the individual emitters where the x-rays are emitted. The variation of the position of the focal point causes a variation of the transmission direction. Thereby, for example, a different transmission angle can be realized in the anterior region than in the molar region. Preferably, a lower focal point is used in the molar region than in the anterior region. The lower focal point in the molar region serves to reduce opposing jaw artifacts. A higher focal point in the anterior region improves the vertical transmission angles, especially in the case of obliquely positioned teeth, and thus leads to an improvement in image quality and diagnosability.

The dental panoramic image could also be generated by a bitewing or temporomandibular joint acquisition, among others.

In one embodiment, the aperture means comprises an aperture per individual x-ray emitter, which can collimate the x-ray beams of the individual x-ray emitters individually. In another embodiment, the aperture means comprises an aperture that can collimate the X-ray beams of all or several individual emitters.

In another embodiment, the aperture means comprises a mechanical aperture that can vary the aperture opening. This can be used to vary the aperture opening during revolution, for example to increase the aperture opening in the anterior tooth region to enhance blurring of structures outside the sharp layer.

In another embodiment, the aperture means comprises a fixed diaphragm.

The emitter array consists of several small, quickly switchable individual emitters, which are spatially distributed. Emitter arrays can be realized using, among others, carbon nanotubes or cold cathode elements as single emitters. This allows the creation of x-ray projections from different projection angles without x-ray-device movement.

The control device can be designed so that the emitted x-ray beams of the at least two individual x-ray emitters differ in the intensity and/or spectral distribution of the x-ray beams in order to produce an intensity and/or spectral distribution that can be varied along the predetermined direction. The extraoral X-ray system can be an X-ray system which can generate panoramic images only, or which can also generate additional DVT images and/or Ceph images.

To generate the panoramic image, the pixels of the panoramic image are calculated from the image pixels of the image series measured by the x-ray detector. The generation of the panoramic image is usually done by summing up the image pixels. This creates the effect that structures in one layer in space are sharply imaged and structures outside this layer are imaged less sharply. By using the two image series under weighting, this effect is enhanced. The weight of an image pixel can also be zero, which corresponds to the omission of the image information of the image pixel.

The generation of the panoramic image is usually done using time stamps, which allow the association between the detector signals and the single emitters used to determine the focus positions and the beam parameters.

The generation of the panoramic image is usually done by using all angle and height information. This offers advantages for the measurement functionalities within the panoramic image, such as improved depth estimation or position determination of structures, as well as the dimensional measurement of specific regions such as caries, lesions and roots. In addition, advantages arise in the visualization of specific regions, such as a layer representation adapted to an occlusal surface, caries, lesion, nerve canal or root.

In a further advantageous embodiment, the computing unit is configured such that the weighting of the image pixels to be applied increases the contrast of the structures to be imaged in a sharp layer in the panoramic image and/or decreases the contrast of the structures to be imaged outside the sharp layer in the panoramic image. A high contrast results from a high relative brightness difference of the imaged structures and clearly defined contours. This has the beneficial effect of improving the representation of the structures to be imaged within the sharp layer of the panoramic image, thus simplifying diagnosis.

In a further advantageous embodiment, the computing unit is configured in such a way that the weighting to be applied is determined relatively lower for image pixels which exhibit a relatively strong absorption of x-rays outside the sharp layer. The mentioned relatively strong absorption can, for example, be attributed to metals in the opposing jaw. Whether a structure is located in the sharp layer can be determined by comparing at least two preliminary panoramic images, because structures in the sharp layer are mapped to a similar position in the preliminary panoramic images. The preliminary panoramic images will be explained in detail in the subsequent description. A low signal at the detector indicates strong absorption. This embodiment has the beneficial effect of improving the representation of the structures to be imaged within the sharp layer of the panoramic image, thus simplifying diagnosis.

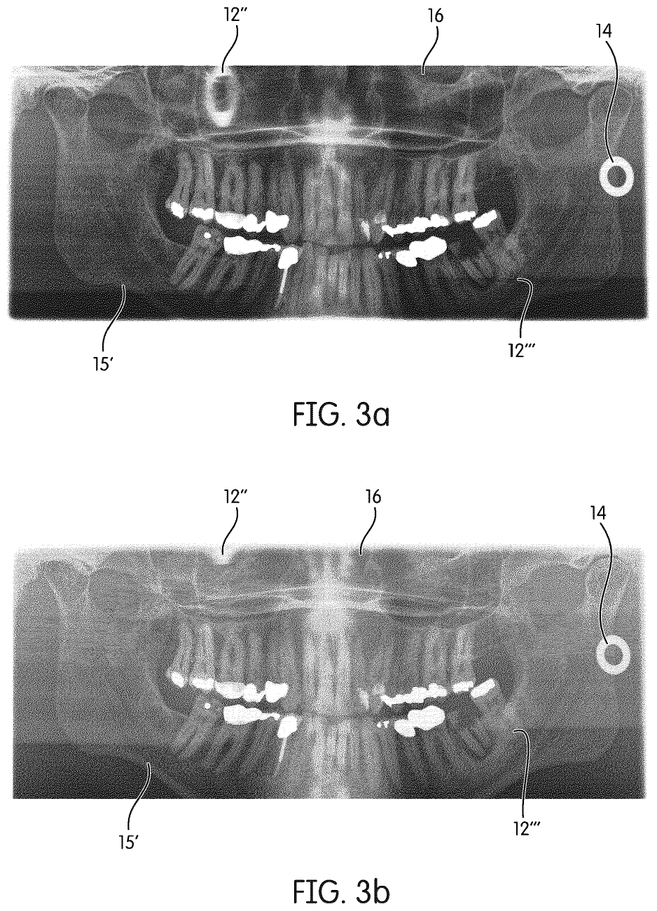

FIGS. 3a and 3b show two preliminary panoramic images (16), respectively reconstructed from series of images from single emitters with different focal heights, showing different positions and characteristics of the opposing jaw artifacts (12″, 12′″). The earring (14) is mapped as an opposing jaw artifact (12″) to a comparatively lower position in FIG. 3a than in FIG. 3b. Similarly, the jawbone (15′) is mapped as an opposing jaw artifact (12′″) in FIG. 3a (see horizontal running shadow transition) to a comparatively lower position than in FIG. 3b. In contrast thereto, the image of the dental structures (e.g., teeth) of the sharp layer remains at the same position in both preliminary panoramic images (16).

In a further advantageous embodiment, the computing unit is configured such that the weighting to be applied is determined to be relatively lower for image pixels which have relatively large differences in absorption of the x-rays along the predetermined direction outside the sharp layer. This is advantageous, for example, in the case of large signal differences at a small local distance, for example to suppress artifacts of the jawbone of the opposing jaw. This has the beneficial effect of improving the representation of the structures to be imaged within the sharp layer of the panoramic image, thus simplifying diagnosis.

In a further advantageous embodiment, the computing unit is configured such that the weighting to be applied is determined on the basis of preliminary panoramic images, each of which is generated from one of the at least two image series and depicts the sharp layer. The generation of the panoramic image can also be realized by weighted computation, for example summation, of the two preliminary panoramic images. This has the advantage that the representation of the structures to be imaged within the sharp layer of the panoramic image is improved compared to the representation in the preliminary panoramic images.

In a further advantageous embodiment, the computing unit is configured such that the weighting to be applied is determined relatively lower for the image pixels which have a relatively different absorption of the x-rays in the corresponding pixels or their local neighborhood of the preliminary panoramic images. If there is a relatively different absorption of the x-rays in the corresponding pixels or their local neighborhood of the preliminary panoramic images, these structures outside the sharp layer are mapped in the panoramic image. Their low weighting has the advantageous effect of an improved representation of the structures to be imaged within the sharp layer of the panoramic image and thus simplifies the diagnosis.

In another advantageous embodiment, the computing unit is configured to determine the applicable weighting of image pixels based on knowledge of the patient anatomy and x-ray device geometry and x-ray device motion retrieved from a storage device. This has the beneficial effect of improving discrimination between structures outside and inside the sharp layer. Thus, this leads to an improved representation of the structures to be imaged within the sharp layer of the panoramic image and simplifies diagnosis.

In a further advantageous embodiment, the computing unit is configured to align the position/shape of the sharp layer to be imaged with the patient's dental structures such as teeth and jaw bones, tooth crowns, tooth roots, tooth root tips, periodontal crevices, nerve canals of jaws and teeth, jaw joints, or dental pathologies of the patient such as caries and inflammations. For the aforementioned alignment of the sharp layer to be imaged, common image optimization methods can be used, including the so-called autofocus function, by calculating it from a series of images, or by calculating it separately for each series of images and then calculating the separate autofocus results together, or by calculating it jointly from the at least two series of images. This has the beneficial effect of improving the representation of the relevant dental structures within the sharp layer of the panoramic image and thus simplifies diagnosis.

In another advantageous embodiment, the computing unit is configured to align or realign the position/shape of the sharp slice to be imaged to the patient's dental structures such as teeth and jaw bones, tooth crowns, tooth roots, tooth root tips, periodontal crevices, nerve canals of jaw and teeth, occlusal surfaces, temporomandibular joints, or dental pathologies of the patient such as caries and inflammations, by using the weighting of the image pixels from at least two image series. In the aforementioned determination of the sharp layer to be imaged, the autofocus function can be used. This has the beneficial effect of improving the visualization of relevant dental structures within the sharp layer of the panoramic image, thus simplifying diagnosis. By using several weighted image series with different projection angles, this leads to an advantageous representation of inclined layers in the panoramic image, such as the representation of occlusal surfaces.

In a further embodiment, multiple panoramic images or partial areas of the panoramic images are generated for which the position of the sharp layer varies in a predetermined area, e.g., in the lingual or buccal direction, using the weighting of the image pixels when generating the panoramic images. This offers the user the possibility to navigate in a narrow range, around the sharp layer, e.g. to show individual roots in the molar region. This enables improved visualization and position determination of the dental structures.

In a further advantageous embodiment, the control device controls the x-ray emitter array and the aperture means in such a way that the said areas to be irradiated are sequentially irradiated by the incident x-ray beams of the at least two individual x-ray emitters. This has the advantageous effect that the respective irradiated areas of the x-ray detector can be read out for each focus position as independently as possible.

In a further advantageous embodiment, the control device drives the at least two individual x-ray emitters sequentially. This has the advantageous effect that the areas to be irradiated are sequentially irradiated by the incident X-ray beams of the at least two individual x-ray emitters.

In a further advantageous embodiment, the control device controls by means of the aperture means to sequentially block the x-rays emitted from the at least two individual x-ray emitters so that an image of the image series is predominantly resulting from the x-rays of one of the at least two individual x-ray emitters. This corresponds to a mechanical, so-called shutter aperture, which sequentially covers all except one of the individual x-ray emitters. This has the advantage that the areas to be irradiated are irradiated sequentially by the incident X-ray radiation of the at least two individual x-ray emitters.

1. An extraoral x-ray system suitable for dental panoramic imaging of a patient, comprising:
an x-ray emitter array having at least two individual x-ray emitters each for emitting x-rays, and offset at least along a predetermined direction;
an x-ray detector for at least partially detecting the x-rays emitted from the at least two individual x-ray emitters during one revolution, wherein the x-ray emitter array and the x-ray detector are arranged for movement relative to each other about an axis running parallel to the predetermined direction, wherein areas of the x-ray detector respectively irradiated by the at least two individual x-ray emitters at least partially overlap;
an aperture for collimating x-ray radiation emitted by the at least two individual x-ray emitters onto the respective areas to be irradiated;
a control device for moving the x-ray emitter array and the x-ray detector about the parallel running axis, and for driving the at least two individual x-ray emitters and for reading out at least two image series of the respective irradiated areas of the x-ray detector during the revolution; and
a computing unit for generating a sharp layer of one panoramic image to be sharply imaged by using the at least two image series, wherein the computing unit is configured to apply a weighting of image pixels when generating the one panoramic image, wherein the image pixels belong to at least the two image series.

2. The extraoral x-ray system according to claim 1, wherein the computing unit is configured such that the weighting of the image pixels to be applied increases a contrast of structures to be imaged in the sharp layer in the panoramic image and/or decreases a contrast of structures to be imaged outside the sharp layer in the panoramic image.

3. The extraoral x-ray system according to claim 1, wherein the computing unit is configured such that the weighting to be applied is determined to be relatively lower for image pixels which have a relatively strong absorption of x-rays outside the sharp layer.

4. The extraoral x-ray system according to claim 1, wherein the computing unit is configured to determine the weighting to be applied relatively lower for image pixels which have relatively strongly different absorptions of the X-rays along the predetermined direction outside the sharp layer.

5. The extraoral x-ray system according to claim 1, wherein the computing unit is configured to determine the weighting to be applied on a basis of preliminary panoramic images, each of which is generated from one of the at least two image series and depicts the sharp layer.

6. The extraoral x-ray system according to claim 5, wherein the computing unit is configured to determine the weighting to be applied relatively lower for the image pixels having a relatively different absorption of the X-rays in corresponding pixels or their local neighborhood of the preliminary panoramic images.

7. The extraoral x-ray system according to claim 1, wherein the computing unit is configured to determine the weighting to be applied to the image pixels based on knowledge of a patient anatomy and an x-ray system geometry and an x-ray system movement to be retrieved from a storage medium.

8. The extraoral x-ray system according to claim 1, wherein the computing unit is configured to align a position of the sharp layer to be imaged with a patient's dental structures such as teeth and jaw bones, tooth crowns, tooth roots, tooth root tips, periodontal crevices, nerve canals of jaws and teeth, temporomandibular joints, or dental pathologies of the patient such as caries and inflammations.

9. The extraoral x-ray system according to claim 1, wherein the computing unit is configured to adapt by using the weighting of the image pixels from at least two image series, a position of the sharp layer to be imaged to a patient's dental structures such as teeth and jaw bones, tooth crowns, tooth roots, tooth root tips, periodontal gaps, nerve canals of jaws and teeth, occlusal surfaces, jaw joints, or dental pathologies, or dental pathologies of the patient such as caries and inflammations.

10. The extraoral x-ray system according to claim 1, wherein a plurality of panoramic images are generated for which a position of the sharp layer varies in a predetermined range, wherein the weighting of the image pixels is used in the generation of the panoramic images.

11. The extraoral x-ray system according to claim 1, wherein the control device, the x-ray emitter array and the aperture are controlled such that the areas to be irradiated are sequentially irradiated by incident x-ray beams of the at least two individual x-ray emitters.

12. The extraoral x-ray system according to claim 11, wherein the control device sequentially drives the at least two individual x-ray emitters.

13. The extraoral x-ray system according to claim 11, wherein the control device, sequentially blocks using the aperture, X-ray radiations emitted from the at least two individual x-ray emitters so that an image of the image series is predominantly resulting from the x-ray radiation of one of the at least two individual x-ray emitters.

14. The extraoral x-ray system according to claim 11, wherein a total height of the irradiated areas along the predetermined direction is greater than a total width along a direction orthogonal thereto by a factor (F) of at least 3 times.
